(19) United States
(12) Patent Application Publication
(10) Pub. No.: US 2020/0188249 A1
(43) Pub. Date: Jun. 18, 2020

(54) COMPOSITION COMPRISING 2-ETHYL-1,3-HEXANEDIOL FOR USE IN A DEODORANT SPRAY OR ANTIPERSPIRANT SPRAY

(71) Applicant: Henkel AG & Co. KGaA, Duesseldorf (DE)

(72) Inventors: BERNHARD BANOWSKI, Duesseldorf (DE); ANGELA MUELLER, Grevenbroich (DE)

(73) Assignee: Henkel AG & Co. KGaA, Duesseldorf (DE)

(21) Appl. No.: 16/669,233

(22) Filed: Oct. 30, 2019

(30) Foreign Application Priority Data
Dec. 18, 2018 (DE) 10 2018 222 058.1

(51) Int. Cl. A61K 8/28; A61K 8/34; A61K 8/27; A61K 8/26; A61K 8/92; A61K 8/25; A61Q 15/00

(52) U.S. Cl. CPC A61K 8/28; A61K 8/345; A61K 8/27; A61Q 15/00; A61K 8/92; A61K 8/25; A61K 8/26

(57) ABSTRACT

The present disclosure relates to an anhydrous cosmetic composition for controlling body odour or underarm wetness, wherein the composition contains a particulate agent to combat odour or underarm wetness and 2-Ethyl-1,3-hexanediol as solvent. The present disclosure further relates to a cosmetic product comprising the anhydrous cosmetic composition and the use of the cosmetic composition.

### CROSS-REFERENCE TO RELATED APPLICATION

This application claims priority to German Patent Application No. 10 2018 222 058.1 filed Dec. 18, 2018, which is incorporated herein by reference in its entirety.

### TECHNICAL FIELD

The present disclosure relates to an anhydrous cosmetic composition for controlling body odour or underarm wetness, wherein the composition contains a particulate agent to combat odour or underarm wetness and a diol as solvent. The present disclosure further relates to a cosmetic product comprising the anhydrous cosmetic composition and the use of the cosmetic composition.

### BACKGROUND

Washing, cleaning and grooming one's body constitutes a fundamental human need. The manufacturers of personal hygiene products are constantly trying to satisfy these human needs in many different ways. Particularly important for daily hygiene is the sustained elimination of body odour. Many body care substances designed specifically to deodorise are known from the related art and have been developed for application in regions of the body with a high density of sweat glands, particularly in the armpit region and on the feet.

Body odour occurs mainly as a result of the bacterial decomposition of individual constituents of sweat on the skin. With regard to deodorising the body a rough distinction can be made between agents which already absorb or conceal unpleasant smelling substances (fragrances, perfumes), and agents which prevent the sweat from being decomposed and therewith the formation of the unpleasant smelling substances, or at least slow the process down (bacteriostatic agents, prebiotically active components and enzyme inhibitors). Depending on the mechanism chosen for preventing body odour, odour absorbers, fragrances, deodorising ion exchangers, bacteriostatic substances, prebiotically acting components, enzyme inhibitors and other active ingredients are usable as cosmetic deodorising agents. The agents must be contained in the compositions, wherein it must be guaranteed that the compositions can be delivered conveniently in a desired administration form, and that the compositions do not cause an unpleasant sensation in the skin.

The cosmetic antiperspirants used to suppress sweat that are described in the related art contain aluminium and/or zirconium compounds as sweat inhibiting agents. Firstly these sweat inhibiting compounds reduce the rate at which the body secretes sweat by temporarily constricting and/or blocking the excretory ducts of the sweat glands, with the result that the quantity of sweat can be reduced by from about 20 to about 60 percent. Secondly, because of their antimicrobial action they have an additional deodorising effect. Moreover, substances are described in the related art which can absorb the unpleasant smelling substances that have already been created. These comprise solids such as activated carbon, zinc ricinoleate, cyclodextrine or all kinds of ion exchangers.

Apart from their mechanism of action, the development of deodorants and antiperspirants is informed by their delivery form. The cosmetic products are assembled and packaged in an enormous assortment of delivery forms, for example as powder, in stick form, as aerosol spray, pump spray, liquid and gel-state roll-on application, as lotion, gel and impregnated flexible substrates (deodorant wipes). Particularly popular with consumers are sprays containing solid particles which are suspended in a liquid matrix. The matrix is often in the form of an oil phase frequently including ethers, triglycerides, ester oils or paraffin substances.

With reference to the development of sprays, product development faces two challenges. Firstly, the organic substances interfere with the release of the agents. These are often soluble in water, and the agents are prevented from dissolving in the sweat on the skin by a film of the organic substances on the agents. Secondly, the organic substances cause unwelcome deposits on textiles.

### BRIEF SUMMARY

Anhydrous cosmetic compositions for controlling body odour or underarm wetness. and cosmetic products including the same, are provided herein. In an embodiment, an anhydrous cosmetic composition includes a particulate agent against odour or underarm wetness and a solvent, wherein the solvent is 2-Ethyl-1,3-hexanediol.

In another embodiment, an anhydrous cosmetic composition for controlling body odour or underarm wetness includes:

a particulate agent against underarm wetness chosen from aluminium chlorohydrate, aluminium-zirconium-chlorohydrate, zinc chloride, zinc sulfocarbolate, zinc sulfate, zirconium chlorohydrate, aluminium-zirconium-chlorohydrate-glycine complexes, silver or a silver salt, or a combination thereof;

2-Ethyl-1,3-hexanediol solvent; and

thickener which is selected from the group of hydrophobically modified clay minerals, hydrophobically modified amorphous silicon dioxide, hydrophilic amorphous silicon dioxide, bentone gels, Versagel types, dextrin esters or a combination thereof.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The problem the present disclosure addresses is that of providing a deodorising and/or antiperspirant composition which enables the active agent to be released more effectively.

A first object of the present disclosure is therefore to provide an anhydrous cosmetic composition for controlling body odour or underarm wetness, wherein the composition contains a particulate agent to combat odour or underarm wetness and a solvent, wherein the solvent is 2-Ethyl-1,3-hexanediol.

Surprisingly it was found that 2-Ethyl-1,3-hexanediol is particularly effective in improving the release of the agent. Moreover, it is possible for the quantity of silicone oils at issue to be reduced and/or their use to be avoided entirely by replacing some of the organic carrier typically used or the entirety thereof with the special solvent.

The compositions as contemplated herein should be anhydrous, in particular substantially anhydrous. The person skilled in the art understands an anhydrous composition to be a composition to which no water is added. Given the fact that raw materials generally—and with regard to the present disclosure particularly the particulate agents—are not completely free of water, the composition as contemplated herein contains water in a small quantity. Moreover, products always absorb a certain amount of moisture. A preferred composition as contemplated herein is substantially free of water. The cosmetic preparation comprises preferably from about 0 to about 3 wt %, more preferably from about 0.1 to about 2 wt %, most preferably from about 0.2 to about 1 wt % water, relative to the total weight of the composition.

It may be assumed that the improved release of the particulate agent or particulate agents is correlated with the combination of the solubility of the solvent in water and its capability to disperse the particulate agents. For this reason, preferred particulate agents are the object of preferred embodiments.

According to a preferred embodiment of the present disclosure, the particulate agent against underarm wetness is an aluminium salt, a zirconium salt, a zinc salt, an aluminium/zirconium mixed salt, preferably aluminium chlorohydrate, aluminium-zirconium-chlorohydrate, zinc chloride, zinc sulfocarbolate, zinc sulfate, zirconium chlorohydrate, aluminium-zirconium-chlorohydrate-glycine complexes, silver or a silver salt, in particular activated aluminium chlorohydrate. Further particularly preferred particulate agents based on silver salts include silver citrate, silver dihydrogen citrate, silver lactate and silver sulfate, soluble complex salts of silver, colloidal silver and silver zeolites.

The object of claim 1 is to be understood to mean that the composition comprises either an agent against underarm wetness or an agent against odour or both. According to a preferred embodiment, the composition also contains a particulate agent against odour as well as or instead of the particulate agent against underarm wetness. According to a preferred embodiment of the present disclosure, the composition comprises a particulate agent against odour which includes one or more of the following: cyclodextrine, zeolites, metal carbonates, preferably zinc carbonate, calcium carbonate, or magnesium carbonate, metal hydroxides, preferably magnesium hydroxide, metal hydrogen carbonates, preferably sodium bicarbonate, mixed salts of alkaline-earth metal hydroxides and alkaline-earth metal carbonate, zinc chloride hydroxide, diatoms, silicates, preferably aluminium silicates, calcium silicate, magnesium-aluminium metasilicate, magnesium-aluminium silicate, sodium-aluminium silicates, magnesium-calcium silicates, or magnesium silicate, silicas, starch or derivatives thereof, aluminium or sodium salts of starch esterified with octenyl succinic anhydride, modified cornstarch, glyceryl starch, amylodextrin, maltodextrin, bentonite, silica, hectorite, kaolin, montmorillonite, perlite, talcum or mixtures of any of the aforementioned, most particularly preferably sodium bicarbonate.

As was noted above, the solvent 2-Ethyl-1,3-hexanediol serves as a substitute for carriers that are usually used, wherein however it is also possible that only a part of the organic solvents usually used is replaced. According to a preferred embodiment of the present disclosure, the composition is based on an organic carrier which is a cosmetic oil comprising liquid paraffin oil or isoparaffin oil, preferably isodecane, isoundecane, isododecane, isotridecane, isotetradecane, isopentadecane, isohexadecane or isoeicosane, esters of linear or branched saturated or unsaturated C2-30 fatty alcohols with linear or branched saturated or unsaturated C2-30 fatty acids, C8-C22 fatty alcohol esters of monovalent or polyvalent C2-C7 hydroxycarboxylic acids, triethyl citrate, branched saturated or unsaturated C6-30 fatty alcohols, mono-, di- and triglycerides of linear or branched saturated or unsaturated, optionally hydroxylated C8-30 fatty acids, dicarboxylic acid esters of linear or branched C2-C10 alkanols, adducts of ethylene oxide and/or propylene oxide with mono- or polyvalent C3-22 alkanols, linear silicone oils, cyclic silicone oils, particularly cyclopentasiloxane, polypropylene glycol ether, polyethylene glycol ether, organic esters—particularly esters of the glycerins, ethers and ether carbonates as well as mixtures of one or more of the above.

According to a preferred embodiment, the composition as contemplated herein contains a thickener to improve the composition's manageability. The thickener is preferably adjusted to the solvent. According to a preferred embodiment of the present disclosure, the composition contains a thickener which is preferably selected from the group consisting of hydrophobically modified clay minerals, hydrophobically modified amorphous silicon dioxide, hydrophilic amorphous silicon dioxide, bentone gels, Versagel types and dextrin esters.

According to a preferred embodiment of the present disclosure, the hydrophobically modified amorphous silicon dioxide is a product of silylation from a reaction between a precipitated silicic acid and/or a pyrogenic silicic acid and an organosilicon compound, wherein the organosilicon compound is preferably an alkylsiloxane, a silane, preferably trimethylsilane, or a silazane, in particular hexamethyl disilazane. The hydrophobically modified amorphous silicon dioxide further comprises preferably alkyl groups with 1 to 18, preferably 2 to 14, more preferably 3 to 12 carbon atoms. More preferably still, the hydrophobically modified amorphous silicon dioxide is Silica Silylate (INCI), Silica Cetyl Silylate (INCI) or Silica Dimethyl Silylate (INCI). The hydrophobically modified amorphous silicon dioxide is rendered hydrophobic by the alkyl groups of the organosilicon compound. After hydrophobisation, the alkyl groups are located on the surface of the silicon dioxide particles. The number of carbon atoms in the alkyl group influences the degree of hydrophobisation. Silica Silylate (INCI), Silica Cetyl Silylate (INCI) and Silica Dimethyl Silylate (INCI) are products which are available commercially. Aerosil R 812 S is a preferred example of such.

Phyllosilicates may also be used as thickeners as an alternative to the hydrophobic silicic acids. According to a preferred embodiment of the present disclosure, clay minerals, preferably hydrophobically modified clay minerals are used, wherein these are preferably selected from montmorillonites, hectorites or bentonite, wherein the hydrophobically modified clay minerals are in particular Disteardimonium Hectorite and Quaternium-18 Hectorite.

According to a preferred composition this contains from about 3 to about 40 wt %, preferably from about 10 to about 30 wt %, more preferably from about 15 to about 25 wt %, particulate agent against underarm wetness, particularly the aluminium salt, and/or from about 1 to about 30 wt %, preferably from about 5 to about 25 wt %, more preferably from about 10 to about 20 wt %, particulate agent against odour, particularly sodium bicarbonate, relative in each case to the total weight of the composition.

According to a further preferred embodiment, the composition contains from about 1 to about 25 wt %, preferably from about 5 to about 20 wt %, more preferably from about 10 to about 15 wt %, of the solvent 2-Ethyl-1,3-hexanediol; from about 0.5 to about 5 wt %, preferably from about 1 to about 4 wt %, more preferably from about 2 to about 3 wt % thickener; and from about 5 to about 85 wt %, preferably from about 20 to about 70 wt %, more preferably from about 40 to about 60 wt % cosmetic oil, relative in each case to the total weight of the composition. In this preferred embodiment, further components may also be contained in the composition. Accordingly, if an exemplary composition contains about 10 wt % solvent, about 2 wt % thickener, about 60 wt % of a cosmetic oil and about 20 wt % sodium bicarbonate, further components are included in order to reach about 100 wt % of the composition.

The advantage effect of wetness reduction is particularly pronounced with the addition of 2-Ethyl-1,3-hexanediol, in particular addition thereof in a quantity from about 5 to about 20 wt %.

Further preferred deodorant additives are selected from “prebiotically active components”, which as contemplated herein are understood to include those components which inhibit only or at least mainly the odour-producing microbes of the skin microflora, and not the desirable microbes, that is to say those which do not produce odour and which are part of a healthy skin microflora. Agents such as conifer extracts, in particular from the group of Pinaceae, and plant extracts from the group of Sapindaceae, Araliaceae, Lamiaceae and Saxifragaceae, particularly extracts of Picea spp., Paullinia sp., Panax sp., Lamium album or Ribes nigrum and mixtures of said substances are named explicitly here.

Further preferred deodorant additives are selected from the perfume oils and Deosafe® perfume oils with bacteriostatic effect which are available commercially from the Symrise company, formerly Haarmann and Reimer.

The sweat inhibiting composition preferably comprises one or more fragrances or odorants. By definition, fragrances or odorants, particularly the fragrances and odorants listed below, are not to be considered together with the cosmetic oils. The definition of an odorant for the purposes of the present application corresponds to the standard technical definition as described in the RÖMPP Chemie Lexikon (“Römpp Lexikon Chemie” [Römpp Chemical Lexicon], J. Falbe, M. Regitz (ed.), 10th edition, Thieme-Verlag). Within the scope of the present disclosure, only substances which constitute about 10 wt % and less of the sweat inhibiting composition—relative to the total weight of the sweat inhibiting composition—may be considered to be fragrances or odorants. Examples of fragrances and odorants of the ester type are benzyl acetate, phenoxyethyl isobutyrate, p-tert.-butylcyclohexyl acetate, linalyl acetate, dimethyl benzyl carbinyl acetate (DMBCA), phenylethyl acetate, benzyl acetate, ethylmethylphenyl glycinate, allyl cyclohexyl propionate, styrallyl propionate, benzyl salicylate, cyclohexyl salicylate, Floramat, Melusat and Jasmecyclat. Examples of fragrances and odorants of the ether type are benzylethyl ether and Ambroxan, examples of fragrances and odorants of the aldehyde type are the linear alkanals with 8-18 C-atoms, citral, citronellal, citronellyl oxyacetaldehyde, cyclamen aldehyde, lilial and bourgeonal, examples of fragrances and odorants of the ketone type are the ionones, alpha-isomethyl ionone and methyl cedryl ketone, examples of fragrances and odorants of the alcohol type are anethole, citronellol, eugenol, geraniol, linalool, phenyl ethyl alcohol and terpineol, examples of fragrances and odorants of the terpene type are limonene and pinene. Examples of fragrances and odorants are pine-, citrus-, jasmine-, patchouli-, rose-, ylang-ylang oil, clary sage oil, camomile oil, clove oil, mint oil, cinnamon leaf oil, linden blossom oil, juniper berry oil, vetiver oil, olibanum oil, galbanum oil, labdanum oil, orange blossom oil, neroli oil, orange peel oil and sandalwood oil, as well as the essential oils such as angelica root oil, anise oil, arnica flower oil, basil oil, bay oil, bergamot oil, champaca flower oil, silver fir oil, silver fir cone oil, elemi oil, eucalyptus oil, fennel oil, spruce needle oil, geranium oil, ginger grass oil, guaiacwood oil, gurjun balsam oil, helichrysum oil, ho oil, ginger oil, iris oil, cajeput oil, calamus oil, camomile oil, camphor oil, cananga oil, cardamom oil, cassia oil, pine needle oil, copaiba balsam oil, coriander oil, spearmint oil, caraway oil, cumin oil, lavender oil, lemongrass oil, lime oil, mandarin oil, melissa oil, musk seed oil, myrrh oil, clove oil, niaouli oil, orange oil, oregano oil, palmarosa oil, patchouli oil, Peru balsam oil, petitgrain oil, pepper oil, peppermint oil, pimento oil, pine oil, rose oil, rosemary oil, sandalwood oil, celery oil, spike oil, star anise oil, turpentine oil, thuja oil, thyme oil, verbena oil, juniper berry oil, wormwood oil, wintergreen oil, hyssop oil, cinnamon oil, citronella oil, lemon oil and cypress oil. Further fragrances and odorants are ambrettolide, alpha-amyl cinnamaldehyde, anethole, anisaldehyde, anisyl alcohol, anisole, anthranilic acid methyl ester, acetophenone, benzyl acetone, benzaldehyde, benzoic acid ethyl ester, benzophenone, benzyl alcohol, benzyl acetate, benzyl benzoate, benzyl formiate, benzyl valerianate, borneol, bornyl acetate, alpha-bromostyrene, n-decyl aldehyde, n-dodecyl-aldehyde, eugenol, eugenol methyl ether, eucalyptol, farnesol, fenchone, fenchyl acetate, geranyl acetate, geranyl formiate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrole, jasmone, camphor, carvacrol, carvone, p-cresol methyl ether, coumarin, p-methoxyacetophenone, methyl-n-amyl ketone, methylanthranilic acid methyl ester, p-methyl acetophenone, methyl chavicol, p-methyl quinoline, methyl-β-naphthyl ketone, methyl-n-nonyl acetaldehyde, methyl-n-nonyl ketone, muscone, β-naphthol ethyl ether, β-naphthol methyl ether, nerol, nitrobenzene, n-nonyl aldehyde, nonyl alcohol, n-octyl aldehyde, p-oxy-acetophenone, pentadecanolide, β-phenyl ethyl alcohol, phenylacetaldehyde dimethyl acetal, phenylacetic acid, pulegone, safrole, salicylic acid isoamyl ester, salicylic acid methyl ester, salicylic acid hexyl ester, salicylic acid cyclohexyl ester, santalol, skatole, terpineol, thymene, thymol, γ-undecalactone, vanillin, veratrum aldehyde, cinnamaldehyde, cinnamyl alcohol, cinnamic acid, cinnamic acid ethyl ester and cinnamic acid benzyl ester. Other (more volatile) odorants are alkyl isothiocyanates (alkyl mustard oils), butanedione, limonene, linalool, linalyl acetate and propionate, menthol, menthone, methyl-n-heptenone, phellandrene, phenyl acetaldehyde, terpinyl acetate, citral and citronellal. Perfumes or perfume oils are particularly preferably contained in the sweat inhibiting composition in a quantity from about 0.05 to about 10 wt %, still more preferably from about 0.1 to about 5 wt %, most preferably from about 0.5 to about 4 wt % relative to the total weight of the sweat inhibiting composition.

The problem addressed by the present disclosure is further solved by the object of claim 9. A second object of the present disclosure is therefore a cosmetic product comprising an anhydrous cosmetic composition as contemplated herein.

Within the scope of the present disclosure, a cosmetic product is understood to refer to an application-ready cosmetic product. According to a preferred embodiment of the present disclosure, the cosmetic product is an aerosol, in particular a spray. In this preferred embodiment, the anhydrous cosmetic composition as contemplated herein is combined with a propellant to expel the anhydrous cosmetic composition. The cosmetic product particularly preferably includes from about 5 to about 70 wt %, preferably from about 10 to about 30 wt % of the anhydrous cosmetic composition and from about 95 to about 30 wt %, preferably from about 90 to about 70 wt % of a propellant relative to the total weight of the cosmetic product, wherein the propellant is most particularly preferably a hydrocarbon, in particular dimethyl ether, propane, butane and/or isobutane.

Alternatively, a preferred cosmetic product is a roll-on applicator. According to a further preferred embodiment, the cosmetic product comprises the anhydrous cosmetic composition which is located in a roll-on receptacle. The cosmetic product is preferably also a roll-on applicator. In further alternatives to preferred embodiments the cosmetic product is also the anhydrous cosmetic composition on a fibre material, in particular a textile, or the cosmetic product comprises the anhydrous cosmetic composition as a lotion composition.

A third object of the present disclosure is therefore the use of an anhydrous cosmetic composition as contemplated herein or a cosmetic product as contemplated herein as aerosol, as roll-on applicator, as lotion, as stick or on a fibre material, in particular a textile, to control body odour and/or underarm wetness.

Of course, features associated with preferred embodiments of the first object of the present disclosure which are described above only with respect thereto, also correspondingly constitute features of preferred embodiments for the second and third objects.

Most particularly preferred compositions as contemplated herein comprise at least one of the following embodiments A) to H):

### A)

Anhydrous cosmetic composition for controlling body odour or underarm wetness, wherein the composition contains a particulate agent against odour or underarm wetness and a solvent, wherein the solvent is 2-Ethyl-1,3-hexanediol and the particulate agent is aluminium chlorohydrate.

### B)

Anhydrous cosmetic composition for controlling body odour or underarm wetness, wherein the composition contains a particulate agent against odour or underarm wetness and a solvent, wherein the solvent is 2-Ethyl-1,3-hexanediol and the particulate agent is sodium bicarbonate.

### C)

Anhydrous cosmetic composition for controlling body odour or underarm wetness, wherein the composition contains a particulate agent against odour or underarm wetness, a solvent and a thickener, wherein the solvent is 2-Ethyl-1,3-hexanediol, the particulate agent is aluminium chlorohydrate and the thickener is the hydrophobically modified amorphous silicon dioxide Silica Silylate (INCI), Silica Cetyl Silylate (INCI) or Silica Dimethyl Silylate (INCI).

### D)

Anhydrous cosmetic composition for controlling body odour or underarm wetness, wherein the composition contains a particulate agent against odour or underarm wetness, a solvent and a thickener, wherein the solvent is 2-Ethyl-1,3-hexanediol, the particulate agent is sodium bicarbonate and the thickener is the hydrophobically modified amorphous silicon dioxide Silica Silylate (INCI), Silica Cetyl Silylate (INCI) or Silica Dimethyl Silylate (INCI).

### E)

Anhydrous cosmetic composition for controlling body odour or underarm wetness, wherein the composition contains a particulate agent against odour or underarm wetness, a solvent and a thickener, wherein the solvent is 2-Ethyl-1,3-hexanediol, the particulate agent is aluminium chlorohydrate and the thickener is the hydrophobically modified amorphous silicon dioxide Silica Silylate (INCI), Silica Cetyl Silylate (INCI) or Silica Dimethyl Silylate (INCI), wherein the solvent is contained in the composition in a quantity from about 5 to about 20 wt % relative to the total weight of the composition.

### F)

Anhydrous cosmetic composition for controlling body odour or underarm wetness, wherein the composition contains a particulate agent against odour or underarm wetness, a solvent and a thickener, wherein the solvent is 2-Ethyl-1,3-hexanediol, the particulate agent is sodium bicarbonate and the thickener is the hydrophobically modified amorphous silicon dioxide Silica Silylate (INCI), Silica Cetyl Silylate (INCI) or Silica Dimethyl Silylate (INCI), wherein the solvent is contained in the composition in a quantity from about 5 to about 20 wt % relative to the total weight of the composition.

### G)

Aerosol comprising an anhydrous cosmetic composition for controlling body odour or underarm wetness and comprising a propellant, wherein the composition contains a particulate agent against odour or underarm wetness, a solvent and a thickener, wherein the solvent is 2-Ethyl-1,3-hexanediol, the particulate agent is aluminium chlorohydrate and the thickener is the hydrophobically modified amorphous silicon dioxide Silica Silylate (INCI), Silica Cetyl Silylate (INCI) or Silica Dimethyl Silylate (INCI), wherein the solvent is contained in the composition in a quantity from about 5 to about 20 wt % relative to the total weight of the composition, wherein the anhydrous cosmetic composition constitutes from about 5 to about 70 wt %, preferably from about 10 to about 30 wt % and the propellant constitutes from about 95 to about 30 wt %, preferably from about 90 to about 70 wt % of the aerosol.

### H)

Aerosol comprising an anhydrous cosmetic composition for controlling body odour or underarm wetness and comprising a propellant, wherein the composition contains a particulate agent against odour or underarm wetness, a solvent and a thickener, wherein the solvent is 2-Ethyl-1,3-hexanediol, the particulate agent is sodium bicarbonate and the thickener is the hydrophobically modified amorphous silicon dioxide Silica Silylate (INCI), Silica Cetyl Silylate (INCI) or Silica Dimethyl Silylate (INCI), wherein the solvent is contained in the composition in a quantity from about 5 to about 20 wt % relative to the total weight of the composition, wherein the anhydrous cosmetic composition constitutes from about 5 to about 70 wt %, preferably from about 10 to about 30 wt % and the propellant constitutes from about 95 to about 30 wt %, preferably from about 90 to about 70 wt % of the aerosol.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. An anhydrous cosmetic composition for controlling body odour or underarm wetness, wherein the composition comprises a particulate agent against odour or underarm wetness and a solvent, wherein the solvent is 2-Ethyl-1,3-hexanediol.

2. The composition according to claim 1, wherein the particulate agent against underarm wetness is chosen from an aluminium salt, a zirconium salt, a zinc salt, an aluminium/zirconium mixed salt, or a combination thereof.

3. The composition according to claim 1, wherein the particulate agent against odour comprises one or more of the following: cyclodextrine, zeolites, metal carbonates, metal hydroxides, metal hydrogen carbonates, mixed salts of alkaline-earth metal hydroxides and alkaline-earth metal carbonates, zinc chloride hydroxide, diatoms, silicates, silicas, starch or derivatives thereof, aluminium or sodium salts of starch esterified with octenyl succinic anhydride, modified cornstarch, glyceryl starch, amylodextrin, maltodextrin, bentonite, hectorite, kaolin, montmorillonite, perlite, talcum or mixtures of any of the aforementioned.

4. The composition according to claim 1, wherein the composition is based on an organic carrier which is a cosmetic oil.

5. The composition according to claim 1, wherein the composition comprises a thickener.

6. The composition according to claim 5, wherein the composition comprises hydrophobically modified amorphous silicon dioxide as the thickener and wherein the hydrophobically modified amorphous silicon dioxide is a product of silylation from a reaction between a precipitated silicic acid and/or a pyrogenic silicic acid and an organosilicon compound.

7. The composition according to claim 6, wherein the hydrophobically modified amorphous silicon dioxide is a product of silylation from a reaction between a precipitated silicic acid and/or a pyrogenic silicic acid and an alkylsiloxane, a silane, or a silazane,

8. The composition of claim 6, wherein the hydrophobically modified amorphous silicon dioxide is Silica Silylate (INCI), Silica Cetyl Silylate (INCI) or Silica Dimethyl Silylate (INCI).

9. The composition of claim 5, wherein the composition comprises hydrophobically modified amorphous silicon dioxide as the thickener and wherein the hydrophobically modified amorphous silicon dioxide comprises alkyl groups with 1 to 18 carbon atoms.

10. The composition according to claim 5, wherein hydrophobically modified clay minerals are present as the thickener and include one or more of Disteardimonium Hectorite or Quatemium-18 Hectorite.

11. The composition according to claim 1, wherein the composition comprises from about 3 to about 40 wt % particulate agent against underarm wetness, and/or from about 1 to about 30 wt % particulate agent against odour, relative in each case to the total weight of the composition, and/or wherein the composition comprises from about 1 to about 25 wt % of the solvent 2-Ethyl-1,3-hexanediol; from about 0.5 to about 5 wt % thickener; and from about 5 to about 85 wt % cosmetic oil relative in each case to the total weight of the composition.

12. The composition according to claim 1, wherein the particulate agent against underarm wetness is chosen from aluminium chlorohydrate, aluminium-zirconium-chlorohydrate, zinc chloride, zinc sulfocarbolate, zinc sulfate, zirconium chlorohydrate, aluminium-zirconium-chlorohydrate-glycine complexes, silver or a silver salt, or a combination thereof.

13. The composition according to claim 1, wherein the particulate agent against underarm wetness comprises activated aluminium chlorohydrate.

14. The composition according to claim 1, wherein the composition is based on an organic carrier which is a cosmetic oil comprising liquid paraffin oil or isoparaffin oil, esters of linear or branched saturated or unsaturated C2-30 fatty alcohols with linear or branched saturated or unsaturated C2-30 fatty acids, C8-C22 fatty alcohol esters of monovalent or polyvalent C2-C7 hydroxycarboxylic acids, triethyl citrate, branched saturated or unsaturated C6-30 fatty alcohols, mono-, di- and triglycerides of linear or branched saturated or unsaturated, optionally hydroxylated C8-30 fatty acids, dicarboxylic acid esters of linear or branched C2-C10 alkanols, adducts of ethylene oxide and/or propylene oxide with mono- or polyvalent C3-22 alkanols, linear silicone oils, cyclic silicone oils, polypropylene glycol ether, polyethylene glycol ether, organic esters, ethers and ether carbonates, as well as mixtures of one or more of the above.

15. The composition according to claim 1, wherein the composition comprises a thickener which is selected from the group of hydrophobically modified clay minerals, hydrophobically modified amorphous silicon dioxide, hydrophilic amorphous silicon dioxide, bentone gels, Versagel types, dextrin esters or a combination thereof.

16. The composition according to claim 1, wherein the composition comprises:
from about 15 to about 25 wt % particulate agent against underarm wetness and/or from about 10 to about 20 wt % particulate agent against odour, relative in each case to the total weight of the composition;
from about 10 to about 15 wt % of 2-Ethyl-1,3-hexanediol;
from about 2 to about 3 wt % thickener; and
from about 40 to about 60 wt % cosmetic oil relative in each case to the total weight of the composition.

17. A cosmetic product comprising an anhydrous cosmetic composition according to claim 1.

18. The cosmetic product according to claim 17, wherein the cosmetic product comprises from about 5 to about 70 wt % of the anhydrous cosmetic composition and from about 95 to about 30 wt % of a propellant relative to the total weight of the cosmetic products.

19. An anhydrous cosmetic composition for controlling body odour or underarm wetness, wherein the composition comprises:
a particulate agent against underarm wetness chosen from aluminium chlorohydrate, aluminium-zirconium-chlorohydrate, zinc chloride, zinc sulfocarbolate, zinc sulfate, zirconium chlorohydrate, aluminium-zirconium-chlorohydrate-glycine complexes, silver or a silver salt, or a combination thereof;
2-Ethyl-1,3-hexanediol solvent;
thickener which is selected from the group of hydrophobically modified clay minerals, hydrophobically modified amorphous silicon dioxide, hydrophilic amorphous silicon dioxide, bentone gels, Versagel types, dextrin esters or a combination thereof.

20. The composition of claim 19, wherein the solvent is contained in the composition in a quantity from about 5 to about 20 wt % relative to the total weight of the composition.
